(19) United States
(12) Patent Application Publication
(10) Pub. No.: US 2026/0263450 A1
(43) Pub. Date: Sep. 10, 2026

(54) USE OF GLYT1 INHIBITOR IN TREATMENT OF ORGAN FIBROSIS

(71) Applicant: TIANJIN MEDICAL UNIVERSITY, Tianjin (CN)

(72) Inventors: Weidong LI, Tianjin (CN); Xuemin LI, Tianjin (CN); Jiali QIU, Tianjin (CN); Ke YANG, Tianjin (CN); Yun FU, Tianjin (CN); Shen LI, Tianjin (CN)

(73) Assignee: TIANJIN MEDICAL UNIVERSITY, Tianjin (CN)

(21) Appl. No.: 19/165,598

(22) PCT Filed: Feb. 29, 2024
(86) PCT No.: PCT/CN2024/079477

§ 371 Date: Sep. 16, 2025

(30) Foreign Application Priority Data
Mar. 17, 2023 (CN) 202310260007.6

(51) Int. Cl. A61K 31/496; A61K 9/00; A61P 11/00

(52) U.S. Cl. CPC A61K 31/496; A61K 9/0053; A61P 11/00

(57) ABSTRACT

The use of a GLYT1 inhibitor in the treatment of organ fibrosis, particularly in the preparation of a drug for treating organ fibrosis, is promising. By targeting GLYT1, the treatment of fibrotic organs is achieved through the inhibition of the glycine transporter GLYT1 via a GLYT1 inhibitor. Drugs containing GLYT1 inhibitors are particularly suitable for treating pulmonary fibrosis. Inhibiting the glycine transporter GLYT1 with a GLYT1 inhibitor reduces collagen synthesis, which can significantly improve bleomycin-induced pulmonary fibrosis and significantly improve the development of pulmonary fibrosis. Furthermore, when GLYT1 is used as the target, the solution has a small influence on other physiological functions.

### CROSS REFERENCE TO THE RELATED APPLICATIONS

This application is the national phase entry of International Application No. PCT/CN2024/079477, filed on Feb. 29, 2024, which is based upon and claims priority to Chinese Patent Application No. 202310260007.6, filed on Mar. 17, 2023, the entire content of which is incorporated herein by reference.

### TECHNICAL FIELD

The present study belongs to the field of medicine and, in particular, to the use of a glycine transporter 1 (GLYT1) inhibitor in the treatment of organ fibrosis.

### BACKGROUND

Organ fibrosis is a pathological change characterized by an increase in fibrous connective tissue and a decrease in parenchymal cells in organ tissues, and it is a common pathological feature of many chronic diseases. Fibrosis can affect any organ. According to statistics, up to 45% of deaths are caused by fibrosis. In addition, common diseases related to fibrosis include liver cirrhosis, hepatitis, nonalcoholic steatohepatitis, chronic kidney disease, myocardial infarction, heart failure, diabetes mellitus, idiopathic pulmonary fibrosis, and scleroderma. Patients with fibrosis-related diseases account for approximately 1/20 of the total number of patients with vital organ diseases. Therefore, the medical burden caused by fibrosis is significant, and approximately ¼ of the global population is directly or indirectly affected by organ fibrosis.

Fibrosis has now become a significant economic burden in healthcare worldwide. Therefore, identifying key therapeutic targets highly associated with human fibrotic diseases and developing efficient antifibrotic therapies targeting these targets are the focus of future research. However, despite substantial progress in understanding the pathology of fibrosis at present, effective treatment methods are lacking.

One of the main approaches for treating organ fibrosis is the use of high-dose glucocorticoids to reduce inflammatory responses, but this approach is associated with relatively severe side effects such as femoral head necrosis and has poor efficacy for treating chronic organ fibrosis caused by nonacute infections. Blocking the transforming growth factor-β (TGF-β) pathway is an important target for treating fibrotic diseases. However, TGF-β signaling is widely distributed in the human body and is involved in crucial physiological functions. Additionally, drugs that act on the Wnt and Notch signaling pathways also have poor selectivity. Drugs for antiorgan fibrosis can also exert therapeutic effects by regulating oxidative stress, lipid metabolism, matrix metalloproteinase (MMP) inhibitors, and other factors.

Pirfenidone and nintedanib have obtained approval from the U.S. Food and Drug Administration (FDA) for the treatment of idiopathic pulmonary fibrosis. Nintedanib is a potent inhibitor of tyrosine kinase receptors such as vascular endothelial growth factor (VEGF), fibroblast growth factor (FGF), and platelet-derived growth factor (PDGF). The mechanism of action of pirfenidone is not yet fully clear; it may exert antifibrotic effects by inhibiting inflammatory mediators such as tumor necrosis factor-α (TNF-α), interleukin-6 (IL-6), interleukin-12 (IL-12), and interleukin-8 (IL-8). Currently, for the treatment of organ fibrosis, in addition to supportive therapy, anti-inflammatory therapy, and stem cell therapy, effective drug treatment methods are still lacking.

### SUMMARY

To address the above technical problems, the present study highlights the use of a GLYT1 inhibitor in the treatment of organ fibrosis.

The technical solutions adopted by the present study are as follows: the use of a GLYT1 inhibitor in the treatment of organ fibrosis.

The use of a GLYT1 inhibitor in the preparation of a drug for treating organ fibrosis.

Preferably, the glycine transporter GLYT1 is inhibited by a GLYT1 inhibitor, and the transfer of glycine, a raw material for collagen synthesis, into cells is reduced, thereby reducing collagen synthesis and effectively reducing the degree of organ fibrosis.

Preferably, the GLYT1 inhibitor RG1678 has the structure shown in formula (1).

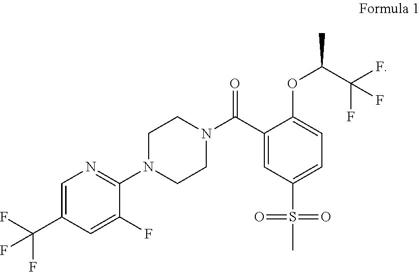 [SMILES: C[C@H](Oc1ccc(S(C)(=O)=O)cc1C(=O)N1CCN(c2ncc(C(F)(F)F)cc2F)CC1)C(F)(F)F]

Preferably, the administration route is oral.

Preferably, it is used for pulmonary fibrosis.

The advantages and positive effects of the present study are as follows: By inhibiting the glycine transporter GLYT1 with a GLYT1 inhibitor, collagen synthesis is reduced, which can significantly improve bleomycin-induced pulmonary fibrosis and significantly improve the development of pulmonary fibrosis. The GLYT1 inhibitor has entered phase II clinical trials for the treatment of malignant tumors, with available phase I clinical safety data. Unlike therapeutic approaches that target pathways such as the TGF-β pathway, this solution has a small influence on other physiological functions.

### BRIEF DESCRIPTION OF THE DRAWINGS

FIG. 1: Body weight changes of the mice in Example 1;

FIG. 2: Changes in the lung weight/body weight ratio of the mice in Example 1;

FIG. 3: Hematoxylin and eosin (HE) staining results of paraffin sections of mouse lung tissue in Example 1;

FIG. 4: Masson staining results of paraffin sections of mouse lung tissue from Example 1;

FIG. 5: Sirius Red staining results of paraffin sections of mouse lung tissue from Example 1;

FIG. 6: Immunohistochemical results of mouse lung tissue sections after bleomycin intervention at a dose of 1 mg/kg in Example 1;

FIG. 7: Immunohistochemical results of mouse lung tissue sections after bleomycin intervention at a dose of 5 mg/kg in Example 1;

FIG. 8: Results of the cytotoxicity evaluation of bleomycin on A549 cells in Example 2;

FIG. 9: Results of the cytotoxicity evaluation of the GlyT1 inhibitor on A549 cells in Example 2.

FIG. 10: Results of detecting pulmonary fibrosis-related indicators by Western blot in Example 2; and

FIG. 11: Results of Western blot grayscale analysis in Example 2.

### DETAILED DESCRIPTION OF THE EMBODIMENTS

In chronic obstructive pulmonary disease (COPD), with the deposition of type I collagen and type III collagen in the extracellular matrix, the elasticity of small airways gradually decreases, accelerating the progression of disease deterioration. In addition, as hepatic stellate cells proliferate, type I collagen and type III collagen gradually replace type IV collagen, and the structure of hepatic sinusoidal capillaries gradually undergoes pathological changes. While changes in vascular structure trigger portal hypertension and related diseases, they further exacerbate the progression of fibrosis. After injury to different parenchymal organs, a strong correlation between the level of collagen deposition in the matrix and the progression of organ fibrosis can be observed. Therefore, collagen deposition has a certain predictive ability for determining the degree of organ fibrosis and evaluating patient prognosis.

The present findings are related to the use of a GLYT1 inhibitor in the treatment of organ fibrosis. The glycine transporter GLYT1 is inhibited by a GLYT1 inhibitor, and the transfer of glycine, a raw material for collagen synthesis, into cells is reduced, thereby reducing collagen synthesis and effectively reducing the degree of organ fibrosis. In some mouse experimental examples of the present study, the GLYT1 inhibitor used was RG1678 (GlyT1 inhibitor 1), the structure of which is shown in formula 1.

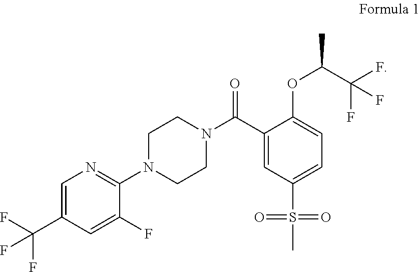 [SMILES: C[C@H](Oc1ccc(S(C)(=O)=O)cc1C(=O)N1CCN(c2ncc(C(F)(F)F)cc2F)CC1)C(F)(F)F]

The dosage was 0.79 mg/kg per day, and the administration route was intragastric gavage. When used, the drug was dissolved in H₂O containing 0.3% Tween 80.

By inhibiting the glycine transporter GLYT1, GLYT1 inhibitors can reduce collagen synthesis, which can significantly improve bleomycin-induced pulmonary fibrosis and have a small influence on other physiological functions. When used for the treatment of pulmonary fibrosis, GLYT1 inhibitors not only have a good effect on the early stage of pulmonary fibrosis but also significantly improve the development of pulmonary fibrosis. GLYT1 inhibitors can be administered orally. The GLYT1 inhibitor used has entered phase II clinical trials and was originally used for the treatment of malignant tumors; its safety is also guaranteed to a certain extent, and it has fewer side effects when used in humans.

The following is an explanation of the solution of the present disclosure in conjunction with the drawings. The experimental methods that do not specifically describe the operation steps are all carried out in accordance with the corresponding product instructions. The instruments, reagents, and consumables used in the examples, unless otherwise specified, can all be purchased from commercial companies.

### Example 1: The GLYT1 Inhibitor RG1678 Alleviates Bleomycin-Induced Pulmonary Fibrosis in Mice

### 1.1 Experimental Animals

C57BL/6J male mice (8-10 weeks old, weighing 24-25 g) were purchased from Beijing Huafukang Biotechnology Co., Ltd. The mice were housed in a specific pathogen-free (SPF) environment, with the room temperature maintained at 20-25° C. and the humidity controlled at 50-60%. The mice were fed normally, and the light was adjusted to a 12 h light/dark cycle. Before modeling, the mice were acclimatized to the corresponding environment for 1 week. All animal experiments were conducted in compliance with the animal care regulations and guidelines of Tianjin Medical University and were approved by the Animal Committee of Tianjin Medical University.

### 1.2 Experimental Materials

Bleomycin (BLM, manufacturer: GLPBIO, USA), RG1678 (GlyT1 Inhibitor 1, manufacturer: Selleck, USA), normal saline, avertin (tribromoethanol, manufacturer: Shanghai Macklin Biochemical Technology Co., Ltd., China), 1 mL syringes, 26½G needles, ophthalmic ointment, medical tweezers, heating lamps, and surgical boards (with an angle of approximately 700 from the horizontal plane) were used.

### 1.3 Experimental Methods: (Bleomycin Administration to Mice Via Oral Instillation)

1. Prediluted bleomycin was prepared, and doses of 1 mg/kg and 5 mg/kg were administered on the basis of the body weights of the mice.

2. Anesthetize each mouse with avertin: Weigh each mouse to calculate the avertin dose (formulated concentration: 1.25%, mouse dose: 0.2 mL/10 g); restrict the mouse's movement by grasping the fur on its neck, and intraperitoneally inject the weight-calculated dose via a 1 mL syringe and a 26½ G needle. The anesthesia duration was 10-40 min.

3. Within 5 min of anesthetic injection, the mice calmed down and stopped moving. The sedation effect was verified by checking for the loss of the righting reflex; if the sedation effect was achieved, the next step was performed.

4. Excessive dryness of the mouse's eyes was avoided during the experiment.

5. The required volume of bleomycin or sterile phosphate-buffered saline (PBS) was loaded into a sterile 200 μL pipette tip.

6. The mouse was placed on the surgical board, and its upper incisors were suspended via a surgical thread loop. Sufficient illumination should be ensured to visualize the vocal cords.

7. The mouse's tongue was gently pulled to one side toward the mandible with sterile padded forceps to expose the vocal cords; then, the pipette tip loaded with bleomycin was lowered to the back of the oral cavity, and the liquid was delivered through the vocal cords during inhalation. The sounds of wheezing were waited to confirm that the liquid was delivered into the trachea. For animals in the control group, an equal volume of sterile PBS was used instead of the bleomycin solution.

8. The tongue was released, and the upper incisors were carefully removed from the suspension thread. The mouse was placed under a heating lamp or on a heating pad until it recovered from anesthesia, which usually occurred within one hour after anesthetic injection.

9. The forceps were cleaned with an alcohol pad before and after each use.

10. The mice were monitored daily until they were euthanized for analysis. RG1678 or water containing 0.3% Tween 80 was added to the mice by gavage every day according to their groupings.

11. When the experiment reached the appropriate time point, the mice were euthanized, their eyeballs were removed for blood collection, and then the mice were dissected to collect samples.

### 1.4 Animal Grouping

Mice in different groups were generated according to the abovementioned methods. The mice in the control group were given normal saline, while the mice in the experimental groups were given bleomycin at different concentrations. Fourteen days after modeling, the mice were intragastrically administered either ddH₂O containing 0.3% Tween 80 or RG1678 prepared in ddH₂O containing 0.3% Tween 80 via continuous gavage for 14 days; the specific experimental conditions are shown in Table 1.

TABLE 1

| Group | Molding | Intragastric Administration |
|---|---|---|
| Control | Normal saline | ddH₂O |
| group 1 | | |
| Experimental | 1 mg/kg bleomycin | ddH₂O |
| group 1 | | |
| Experimental | 1 mg/kg bleomycin | RG1678 |
| group 2 | | |
| Experimental | 5 mg/kg bleomycin | ddH₂O |
| group 3 | | |
| Experimental | 5 mg/kg bleomycin | RG1678 |
| group 4 | | |

### Drug Preparation:

1. Bleomycin stock solution (10 mg/mL): First, 10 mg of bleomycin was weighed and dissolved in 1 mL of 0.9% sodium chloride. The instillation volume for each mouse was calculated on the basis of its body weight, with the instillation volume per mouse being approximately 50 μL. The stock solution was diluted to 0.5 mg/mL and 2.5 mg/mL for subsequent instillation according to the different groups (1 mg/kg and 5 mg/kg).

2. 0.3% Tween 80: Mix 30 μL of Tween 80 with 9970 L of ddH₂O to prepare 10 mL of ddH₂O containing 0.3% Tween 80.

3. RG1678 (0.79 mg/mL): An appropriate amount of RG1678 was dissolved in ddH₂O containing 0.3% Tween 80 to prepare a stock working solution of 0.79 mg/mL. The intragastric administration volume was then calculated on the basis of the mouse's body weight, with approximately 200 μL per mouse. Before use, the stock solution was diluted to a working concentration of 0.079 mg/mL.

After administration via the airway, the mice were observed to immediately exhibit tachypnea, and rales could be heard when they listened closely. After regaining consciousness from anesthesia, the mice in the BLM group presented significantly delayed responses, huddled together, trembled, and developed cyanosis at the extremities of their limbs; subsequently, their food intake decreased, their body weight gradually decreased, and their skin became wrinkled. The mice in the BLM+RG1678 group presented milder degrees of the aforementioned changes, with a less pronounced decrease in body weight.

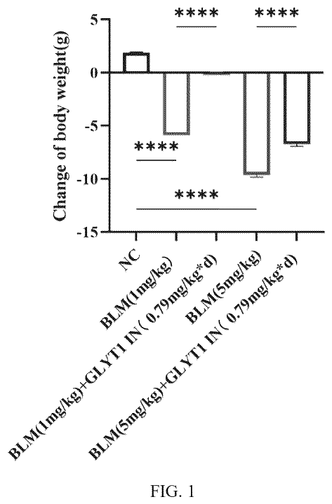

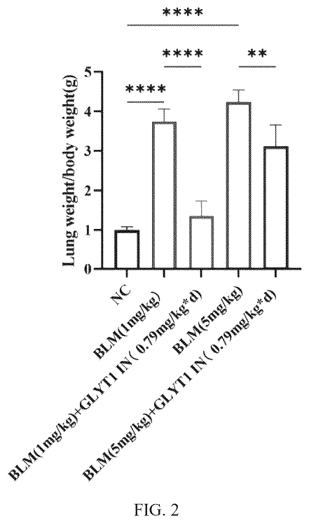

The body weights of the mice were measured before modeling, and their body weights were monitored daily during the experiment to observe changes. A comparison was made between the body weight at the end of the experiment and that at the beginning. As shown in FIGS. 1-2, compared with those in the NC group, the body weights of the mice in the BLM group significantly decreased in a dose-dependent manner (****P<0.0001), even dropping below their initial body weight, and the lung weight/body weight ratio increased significantly (*P<0.05); this increase also showed a dose-dependent pattern. In contrast, compared with those in the BLM group, the body weights of the mice in the BLM+GLYT1 IN group were significantly greater, and the change in the lung weight/body weight ratio was significantly alleviated (**P<0.01, ***P<0.001).

To further observe lung tissue damage in mice in detail, pathological detection was performed on the mouse lung tissues in this example. After HE staining, observation under a light microscope revealed that, as shown in FIG. 3, the tissue structure of the NC group was roughly normal, with clear alveolar structures. After BLM stimulation, the lung tissue structure of the mice was significantly disorganized, the alveolar structures disappeared, the alveolar septa thickened, vascular congestion was obvious, diffuse exudation and infiltration of inflammatory cells were observed, and the lung tissue exhibited consolidation and obvious fibrosis. When RG1678 (GlyT1 inhibitor 1) intervention was initiated 14 days after BLM modeling, it also reduced BLM-induced lung tissue consolidation and significantly alleviated the degree of fibrosis.

Masson staining was performed on mouse lung tissue sections. Masson staining is an experimental method used to detect the secretion and deposition levels of collagen fibers in tissues. After treatment with different staining solutions, the overall background of the tissue section appeared red, the cell nuclei were black, and the collagen fibers were blue. The content of collagen fibers can be evaluated on the basis of the depth of the blue color and the scope of its distribution.

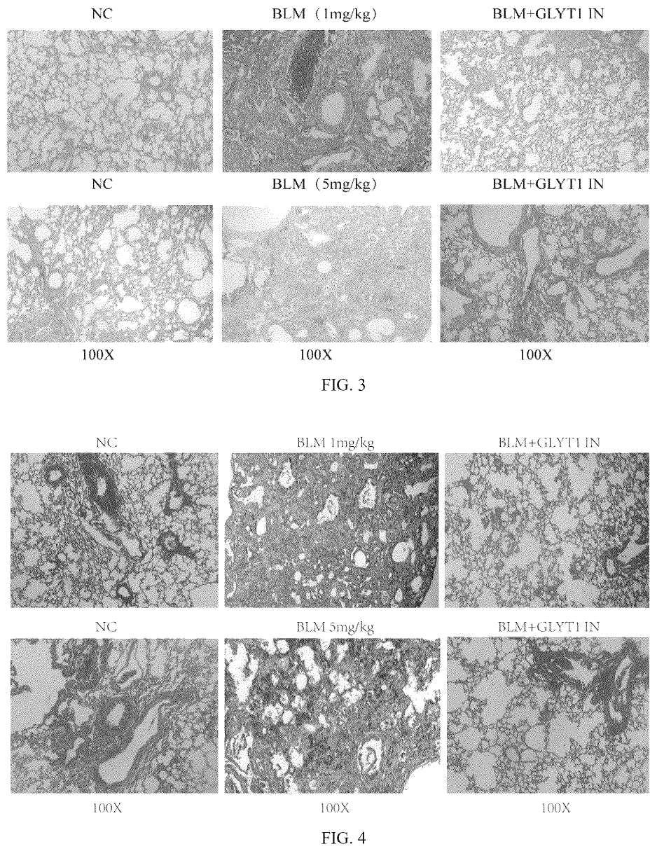

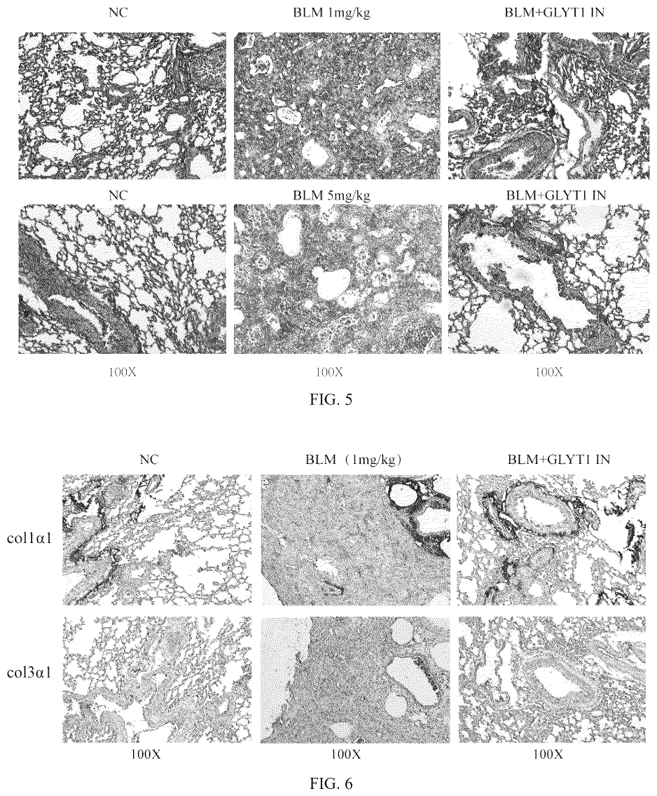

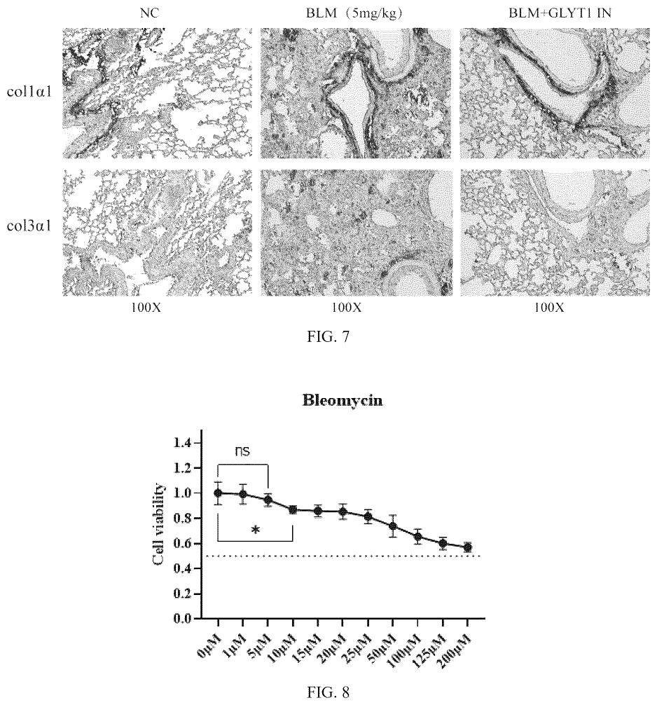

As shown in FIG. 4, compared with that in the NC group, a large amount of collagen deposition was observed in the lung tissue of the mice in the BLM group. This deposition was very obvious in both the 1 mg/kg and 5 mg/kg BLM groups and was diffuse, accompanied by significant lung consolidation and the disappearance of alveolar structures. In contrast, the collagen deposition in the BLM+GLYT1 IN group was not obvious, which was significantly different from that in the BLM group; the alveolar structures were well preserved, and there were no obvious lung consolidation-like changes. In the bleomycin-induced mouse pulmonary fibrosis model, a large amount of collagen is secreted in mouse lung tissue, and intervention with GlyT1 inhibitor 1 can significantly reduce bleomycin-induced collagen deposition in mouse lung tissue.

Sirius Red staining was performed on mouse lung tissue sections. Sirius Red staining is also an experimental method for detecting the secretion and deposition levels of collagen fibers in tissues. The basic groups in collagen fibers bind to the strongly acidic Sirius Red staining solution. Under a conventional optical microscope, cell nuclei appear blue, whereas collagen appears red; under a polarizing microscope, a birefringence phenomenon occurs, which allows differentiation between type I and type III collagen fibers.

As shown in FIG. 5, the results again revealed that there was no obvious collagen deposition in the lung tissue sections of the mice in the NC group, the alveolar structure was relatively clear, and slight collagen deposition was observed around the bronchi. After BLM stimulation, obvious collagen deposition with diffuse distribution was observed in the lung tissue sections of the mice. After intervention with GlyT1 inhibitor 1, collagen deposition was significantly reduced, and the alveolar structure was relatively normal and clear; however, slight collagen deposition was still observed around the bronchi. This effect was achieved when the intervention was initiated 14 days after modeling.

To further analyze the effect of GlyT1 inhibitor 1 on collagen after pulmonary fibrosis modeling, we performed immunohistochemical analysis on mouse lung tissue sections to detect the expression levels of type I collagen and type III collagen. Compared with those in the NC group, the deposition of type I collagen and type III collagen in mouse lung tissue was obvious after BLM intervention, with high expression levels. After 14 days of modeling, GlyT1 inhibitor 1 was administered for treatment. After 14 days, the deposition of type I collagen and type III collagen was significantly lower than that in the BLM group, and their expression levels also decreased significantly.

### Example 2: GlyT1 Inhibitor 1 Alleviates Bleomycin-Induced A549 Cell Damage and its Mechanism

### 2.1 A549 Cells

Bleomycin (BLM, manufacturer: MedChemExpress, USA); GlyT1 inhibitor (GLYT1 IN, manufacturer: MedChemExpress, USA); risperidone (RIS, manufacturer: MedChemExpress, USA); COL1A1 primary antibody (ABclonal Technology Co., Ltd., China); COL3A1 primary antibody (ABclonal Technology Co., Ltd., China); COL4A1 primary antibody (ABclonal Technology Co., Ltd., China); β-actin primary antibody (ABclonal Technology Co., Ltd., China); epithelial cadherin (E-cadherin) primary antibody (ABclonal Technology Co., Ltd., China); and alpha-smooth muscle actin (α-SMA) primary antibody (ABclonal Technology Co., Ltd., China) were used.

### 2.2 Cell Proliferation and Cytotoxicity Analysis (Cell Counting Kit-8 (CCK-8))

A CCK-8 kit was used to detect cell proliferation and cytotoxicity. Its working principle is as follows: In the presence of an electron coupling reagent, WST-8 (chemical name: 2-(2-methoxy-4-nitrophenyl)-3-(4-nitrophenyl)-5-(2,4-disulfophenyl)-2H-tetrazolium monosodium salt) can be reduced by mitochondrial dehydrogenases to produce a highly water-soluble orange-yellow formazan product. The intensity of the color is proportional to cell proliferation and inversely proportional to cytotoxicity. For the same type of cells, the color intensity shows a linear relationship with the number of cells. The OD value measured at a wavelength of 450 nm via a microplate reader can indirectly reflect the number of viable cells.

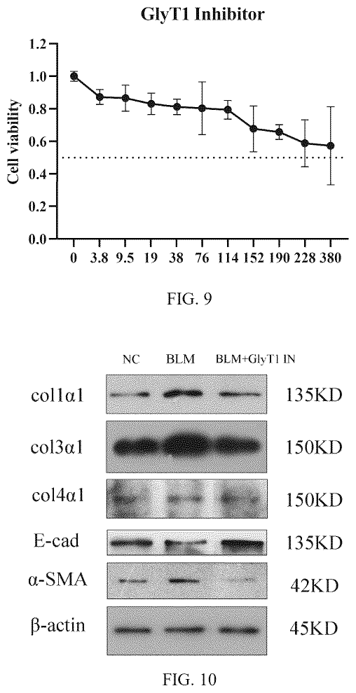

The CCK-8 assay was used to detect the viability of A549 cells after treatment with different concentrations of BLM and GlyT1 inhibitor for 72 h. As shown in FIGS. 8-9, the viability of A549 cells decreased with increasing concentrations of BLM or GlyT1 inhibitor, and stimulation with 10 μM bleomycin significantly affected viability. In this example, BLM, which maintained the viability of A549 cells at approximately 80%, was selected for stimulation, and A549 cells were stimulated with 25 μM BLM to establish the cell model. With increasing concentrations of GLYT1 IN, the viability of A549 cells gradually decreased. At a concentration of 380 nM, the viability of A549 cells was greater than 50%. To obtain stable experimental results more easily, 190 nM GLYT1 IN was selected as the treatment concentration in subsequent studies.

### 2.3 Effects of GlyT1 Inhibitors on Alveolar Epithelial Cells Under Bleomycin-Induced Damage

A bleomycin-induced pulmonary fibrosis model was established in vitro, with simultaneous intervention via a GLYT1 inhibitor.

For the in vitro bleomycin-induced pulmonary fibrosis model, A549 cells were divided into four treatment groups: the NC group (control group: treated with normal saline), the BLM group (bleomycin-treated group: treated with 25 μM BLM for 72 h), and the BLM+GLYT1 IN group (bleomycin and glycine transporter 1 inhibitor-treated group: treated with 25 μM BLM for 72 h and 190 nM GLYT1 IN for 72 h).

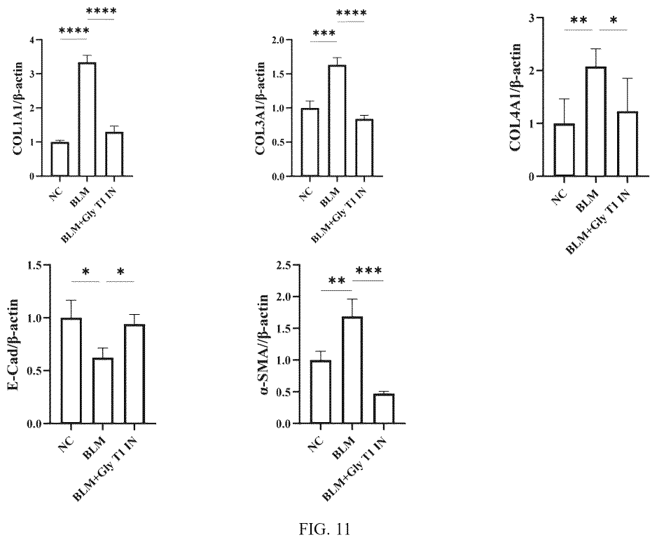

Western blotting was used to detect the expression levels of pulmonary fibrosis-related indicators: collagen type I alpha 1 (col1α1), collagen type III alpha 1 (col3α1), collagen type IV alpha 1 (col4α1), E-cadherin (E-Cad), and α-SMA proteins. As shown in FIGS. 10-11, after BLM stimulation, the protein expression levels of col1α1, col3α1, col4α1, and α-SMA increased significantly, whereas the protein expression level of E-cadherin decreased significantly. Compared with the BLM group, the GlyT1 inhibitor intervention group presented significantly decreased expression levels of the col1α1, col3α1, col4α1, and α-SMA proteins and significantly increased expression levels of the E-cadherin protein. These results indicate that the GlyT1 inhibitor can indeed improve the cell-level changes induced by bleomycin, suggesting that it has a genuine ameliorative effect on pulmonary fibrosis.

On the basis of the results of the above cell and animal models, it can be concluded that GlyT1 inhibitor 1 can significantly improve organ fibrosis.

The embodiments of the present disclosure have been described in detail above, but the content is only the preferred embodiment of the present disclosure and should not be considered as limiting the scope of implementation of the present disclosure. All equivalent changes and improvements made in accordance with the scope of the present disclosure application shall still fall within the patent coverage of the present disclosure.

What is claimed is:

1. A method for treating organ fibrosis, comprising using a glycine transporter 1 (GLYT1) inhibitor.

2. (canceled)

3. The method according to claim 1, wherein glycine transporter GLYT1 is inhibited by the GLYT1 inhibitor, and a transfer of glycine, a raw material for collagen synthesis, into cells is reduced, thereby reducing collagen synthesis and effectively reducing a degree of organ fibrosis.

4. The method according to claim 1, wherein the GLYT1 inhibitor is RG1678 with a structure shown in formula 1;

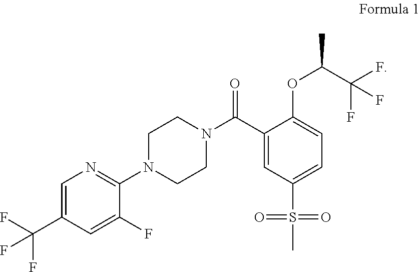 [SMILES: C[C@H](Oc1ccc(S(C)(=O)=O)cc1C(=O)N1CCN(c2ncc(C(F)(F)F)cc2F)CC1)C(F)(F)F]

5. The method according to claim 4, wherein an administration route is oral.

6. The method according to claim 1, wherein the GLYT1 inhibitor is used for treating pulmonary fibrosis.
